(19) United States
(12) Patent Application Publication
(10) Pub. No.: US 2025/0367413 A1
(43) Pub. Date: Dec. 4, 2025

(54) GUIDEWIRE FOR PREVENTION OF RETENTION AND METHODS OF USE AND MANUFACTURE THEREOF

(71) Applicant: The George Washington University, Washington, DC (US)

(72) Inventors: Andrew Meltzer, Washington, DC (US); Daniel I. Shpigel, Washington, DC (US); Aditya Loganathan, Washington, DC (US)

(21) Appl. No.: 19/224,405

(22) Filed: May 30, 2025

Related U.S. Application Data
(60) Provisional application No. 63/655,512, filed on Jun. 3, 2024.

(51) Int. Cl. A61M 25/09

(52) U.S. Cl. CPC A61M 25/09041; A61M 2025/09125

(57) ABSTRACT

Provided herein is a guidewire for preventing guidewire retention. The guidewire can include an elongated wire and a stopping mechanism. The elongated wire can include an insertion portion and an external portion. The stopping mechanism can be located along the external portion. The stopping mechanism can be configured to prevent the external portion from entering a blood vessel of a patient.

### CROSS-REFERENCE TO RELATED APPLICATIONS

This application claims priority benefit to U.S. Provisional Application No. 63/655,512, filed Jun. 3, 2024, the contents of which are entirely incorporated by reference herein.

### FIELD OF DISCLOSURE

The present disclosure relates to guidewires and methods of use and manufacture thereof.

### BACKGROUND

Guidewires are used in medical procedures to guide various medical devices and medications to a target site within a patient. For example, guidewires are inserted into a blood vessel using the Seldinger technique. The Seldinger technique requires using a hollow needle to puncture a patient's skin and underlying structures such that the tip of the needle is located in the target structure. With one hand the needle is stabilized in the target structure. With the second hand, the medical practitioner threads the guidewire into the outer side of the hollow needle and into the target structure, such that a portion of the guidewire is located inside the target structure and a portion is located outside the skin and accessible to the medical practitioner. Next, the medical practitioner uses one hand to maintain the position of the guidewire and another hand to remove the needle from the patient, which requires manipulation of the hands such that there is consistently a hand preventing the dislodgement of the guidewire. Further, one or more instruments can be threaded along the guidewire by the medical practitioner while the medical practitioner holds the guidewire. However, if the guidewire is not properly secured by the medical practitioner, the guidewire can displace into the patient, thereby causing significant medical injuries to the patient. Other techniques for inserting guidewires also require securement of the guidewire by the medical practitioner.

Therefore, there is a need for a guidewire that maintains a desired position without the need to be held by a medical practitioner.

### SUMMARY

Provided herein is a guidewire for preventing guidewire retention. The guidewire can include an elongated wire and a stopping mechanism. The elongated wire can include an insertion portion and an external portion. The stopping mechanism can be located along the external portion. The stopping mechanism can be configured to prevent the external portion from entering a blood vessel of a patient.

In some aspects, the stopping mechanism can include a bend. In some aspects, the bend can have a bend angle between the insertion portion and the external portion. In some aspects, the bend angle can be at least 45 degrees. In some aspects, the bend angle can be at least 90 degrees. In some aspects, the bend can have a bend radius of greater than about 0.75 inches. In some aspects, the bend radius can be less than about 2.50 inches. In some aspects, the bend can be heat treated. In some aspects, the stopping mechanism can include a coiled section of the elongated wire. In some aspects, the stopping mechanism can include an expansion mechanism, an adhesive mechanism, and/or a magnetic mechanism attached to the elongated wire. In some aspects, the stopping mechanism can include a solid ring mechanism operable to receive the elongated wire and secure to a location on the external portion. In some aspects, the elongated wire can include a stainless steel wire. In some aspects, the elongated wire can include nitinol.

Further provided herein is a method of manufacturing a guidewire. The method can include providing the guidewire including an elongated wire and forming a bend in the guidewire by wrapping the elongated wire around a cylinder. The bend can have a bend angle of 45 degrees or greater and a bend radius of between about 0.75 inches and 2.50 inches. The elongated wire can include an insertion portion and an external portion. The bend can be located along the external portion. The bend can prevent the external portion from entering a blood vessel of a patient.

In some aspects, the bend radius can be about 1 inch. In some aspects, the bend angle can be 90 degrees or greater. In some aspects, forming the bend can include heating the bend. In some aspects, the elongated wire can be wrapped around the cylinder at a formation bend radius. In some aspects, the formation bend radius can be less than the bend radius, thereby allowing the elongated wire to bounce back to the bend radius.

### BRIEF DESCRIPTION OF FIGURES

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIGS. 1A-1D illustrate guidewires in various examples.

FIG. 2A illustrates a guidewire in one example.

FIG. 2B illustrates a guidewire in one example.

FIG. 3 illustrates a guidewire in one example.

FIGS. 4A-4C illustrate a stopping mechanism in one example.

FIG. 5 illustrates a stopping mechanism in one example.

FIG. 6 illustrates a guidewire in one example.

FIG. 7 illustrates two examples of packaged guidewires.

FIG. 8 illustrates a test apparatus for testing a guidewire in one example.

FIG. 9 illustrates a test apparatus for testing a guidewire in one example.

FIG. 10 illustrates a test apparatus for testing a guidewire in one example.

FIG. 11 illustrates a test apparatus for testing a guidewire in one example.

FIGS. 12A-12B illustrate a guidewire gravity test in one example.

FIGS. 13A-13B illustrate a tensile strength test for a guidewire in one example.

FIG. 14 illustrates a flowchart of a method for manufacturing a guidewire in one example.

FIG. 15 illustrates a flowchart of a method for using a guidewire in one example.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, +0.5-1%, +1-5% or +5-10% of the recited value, that one would consider equivalent to the recited value, for example, having the same function or result.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Guidewires are thin, flexible medical devices used to guide other instruments (e.g., catheters) within a patient's body. Guidewires are typically sterilized before use and are used in sterile procedures. In some examples, guidewires can be used in non-sterile procedures. Guidewires are typically single-use (e.g., only used in a single procedure). The guidewire provided herein can be utilized in substantially the same procedures and in substantially the same manner as commercially-available guidewires.

Guidewires can be located and guided within a patient's body using a variety of techniques. However, the techniques for guidewire insertion require detailed manipulation of a medical practitioner's hands and carry risks of the guidewire slipping entirely into the patient's body. For example, guidewires can be located within the patient's body using the Seldinger technique. The Seldinger technique requires a medical practitioner to first insert a hollow needle into the patient and locate the needle at a target site within the patient. The medical practitioner then secures the hollow needle with one hand and threads the guidewire into the hollow needle to the target site with a second hand. This technique requires detailed manipulation of the hands, such that the guidewire does not slip into the patient. While the Seldinger technique is used as an example of a guidewire insertion technique, it will be appreciated that the guidewire described herein can be used with other guidewire insertion techniques known in the art.

After the guidewire is properly located at the target site, the hollow needle can be removed while the medical practitioner holds the guidewire. Further, after the hollow needle is removed, various medical instruments can be passed over the guidewire to the target site. For example, dilators, catheters, and other instruments such as tubes, drains, and other medical devices can be passed over the guidewire and subsequently removed or fixed in place. During these procedures, the medical practitioner continues to hold the guidewire such that it does not displace into the patient. A high degree of dexterity and coordination is required for manipulation of the guidewire to both maintain its position with one hand and thread instruments over the wire and into the patient with another hand, which may involve varying degrees of resistance and varying instrument size and rigidity.

If the guidewire is not securely held by the medical practitioner, the guidewire can be drawn into the body of the patient. For example, when the guidewire is located in a blood vessel, the pressure and force exerted on the guidewire by the patient's blood flow can cause the guidewire to be pulled into the patient in the direction of the blood flow. This issue is known as guidewire embolization or retention, which can cause severe medical complications. In some examples, when the guidewire slips into the patient, surgical procedures must be conducted to remove the guidewire from the patient. The guidewire can damage the blood vessels of the patient and/or cause other medical complications.

Provided herein are guidewires, methods of manufacturing guidewires, and methods of using guidewires. The guidewires provided herein remove the need for a medical practitioner to hold the guidewire while the guidewire is at a target site within the patient. For example, the guidewires described herein utilize a stopping mechanism operable to maintain an exterior portion of the guidewire outside of the patient's body. In this manner, embolization and/or retention of the guidewire is prevented. Further, the medical practitioner is provided with significantly improved mobility, as the medical practitioner is no longer required to hold the guidewire while instruments are passed along the guidewire and/or when the hollow needle (e.g., insertion needle) is removed from the target site over the guidewire.

The guidewire described herein can be used in any type of medical procedure that utilizes a guidewire. For example, the guidewire described herein can be used in cardiac catheterization, percutaneous transluminal angioplasty, central venous catheterization, coronary angiography, vascular access and interventions (e.g., neurovascular, cardiovascular, thoracic, etc.), urological interventions, gastrointestinal procedures, interventional radiology, and other procedures where a guidewire is used. It will be appreciated that the preceding list of procedures is exemplary only and that the guidewire described herein can be used in any medical procedure where a guidewire is used.

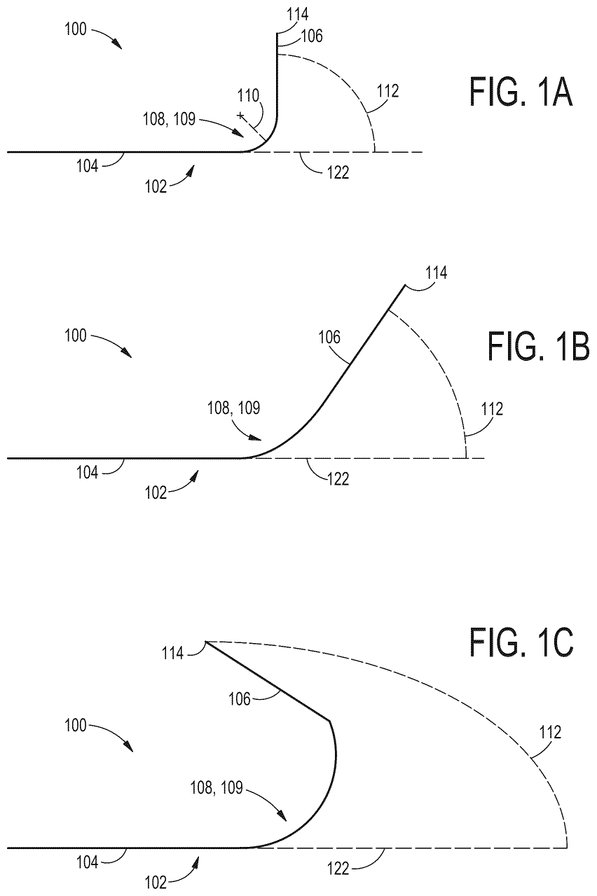

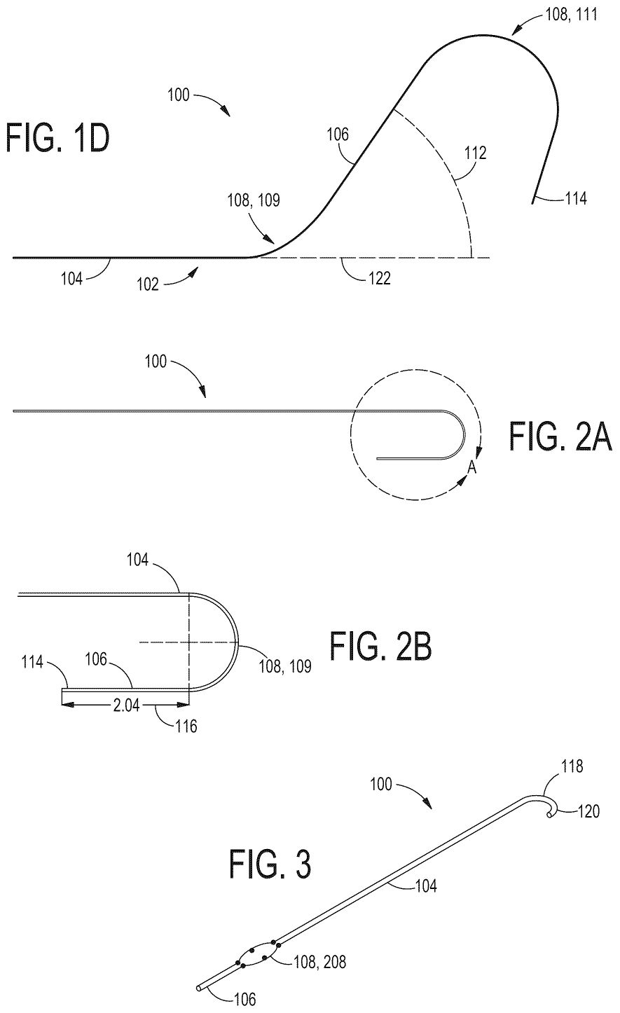

FIGS. 1A-1D and 2A-2B illustrate a guidewire 100. The guidewire 100 can be operable to prevent guidewire retention and/or embolization. The guidewire 100 can include an elongated wire 102. The elongated wire 102 can include an insertion portion 104 and an external portion 106. The insertion portion 104 can be operable to be inserted into a blood vessel of a patient. For example, the insertion portion 104 can be inserted into a blood vessel of a patient by threading the insertion portion 104 through a hollow needle which is located in the target blood vessel. In some examples, the external portion 106 is operable to remain outside of the patient during use. The insertion portion 104 and external portion 106 can be operable to receive one or more instruments. For example, a user can thread one or more instruments over the external portion 106 and deliver the one or more instruments to a target site within the blood vessel by passing the one or more instruments over the insertion portion 104.

In some examples, the elongated wire 102 can be a stainless steel core guidewire. In some examples, the elongated wire 102 can be a nitinol wire. In other examples, the elongated wire 102 can include a stainless steel portion and a nitinol portion. For example, the insertion portion 104 can be stainless steel and the external portion 106 can be nitinol. In other examples, the elongated wire 102 can be formed from other materials commonly used for guidewires. In some examples, the elongated wire 102 can include a stainless steel core with a flat-wire coil and polytetrafluoroethylene (PTFE) coating. In some examples, the elongated wire 102 can include a nitinol wire braided with stainless steel. In some examples, the elongated wire 102 can include solid steel or nitinol core wires and a solid core wire wrapped in a smaller wire coil or braid. In some examples, the elongated wire 102 can include a nitinol tube with micro-cut slots instead of braided wire to improve torque control. In some examples, the elongated wire 102 can be coated with a polymer, such as silicone or polytetrafluorethylene (PTFE) to increase lubricity. In some examples, the elongated wire 102 can include nickel-titanium, a nickel-cobalt alloy, and/or a Hastelloy. In some examples, the elongated wire 102 can be any type of wire used for guidewires.

In some examples, a distal end 118 of the elongated wire 102 can include a J-hook 120. In some examples, the J-hook 120 can be operable to allow for atraumatic passage through blood vessels and prevent complications such as perforation of blood vessels or entrapment.

The guidewire 100 can further include a stopping mechanism 108. In some examples, the stopping mechanism 108 can be located along the external portion 106 of the elongated wire 102. For example, the stopping mechanism 108 can be formed as part of the external portion 106. In some examples, the stopping mechanism 108 can be attached to the external portion 106. The stopping mechanism 108 can be operable to maintain the external portion 106 of the guidewire 100 outside of a patient during use. For example, various forces may act on the insertion portion 104 of the guidewire 100 when the insertion portion 104 is within a blood vessel of a patient. In some examples, the blood flow of the patient can provide a force to the insertion portion 104, thereby pulling the guidewire 100 inward. The stopping mechanism 108 can be operable to overcome the force provided by the blood flow of the patient such that the external portion 106 remains outside of the patient's body during use. The stopping mechanism 108 can significantly simplify the use of a guidewire 100 by no longer requiring a user to hold the external portion 106 of the guidewire 100 to overcome the forces on the insertion portion 104.

In some examples, the stopping mechanism 108 can be located along the external portion 106 such that the insertion portion 104 remains in the target site. For example, the stopping mechanism 108 can be located at a specific length from a distal end of the insertion portion 104 (e.g., the insertion portion 104 can have a length). The length of the insertion portion 104 can be chosen based on an operation type and linear distance from the skin of the patient to the target site.

In some examples, the stopping mechanism 108 can be a bend 109 formed in the elongated wire 102, as illustrated, for example, in FIGS. 1A-1D and 2A-2B. The bend 109 can be operable to overcome the forces applied to the insertion portion 104 during use of the guidewire 100 in the patient. In some examples, the bend 109 can be a permanent bend. The bend 109 can be formed in the external portion 106 of the guidewire 100 using the manufacturing techniques described herein. In some examples, the bend 109 is operable to maintain substantially the same shape during use (e.g., not flexible, rigid, or semi-flexible). In some examples, the bend 109 itself is semi-flexible but automatically biases back to the bend shape when not provided a significant force (e.g., a force substantially greater than the forces applied to the insertion portion 104 by the blood flow of the patient). For example, the bend 109 can be a self-biasing bend such that it reverts back to its shape when not provided a significant force (e.g., a force much greater than the force experienced by the guidewire within the patient's body). By maintaining some flexibility in the bend 109, one or more instruments can be easily passed along the external portion 106, through the bend 109, and to the insertion portion 104 for delivery to the target site. In some examples, the bend 109 can maintain its shape during use, but can be unbent after use by supplying a significant force (e.g., a force much greater than the force experienced on the guidewire 100 within the patient's body) to the bend 109.

In some examples, the bend 109 can have a bend radius 110 and a bend angle 112 configured to overcome the forces applied to the insertion portion 104 during use of the guidewire 100 (e.g., forces provided by blood flow of the patient or other forces exerted on the wire). For example, the bend radius 110 and the bend angle 112 can be configured to overcome a force provided by a blood flow rate of at least 45 cm/sec or greater and a pressure of at least 8 mmHg (696 Pascals) or greater. In some examples, the bend radius 110 and the bend angle 112 can overcome any force acting on the guidewire 100 within the patient's body. For example, the bend radius 110 and the bend angle 112 can overcome a force provided by a blood flow rate of up to 45 cm/sec or more and a pressure of up to 8 mmHg (696 Pascals) or more. In some examples, the bend radius 110 and the bend angle 112 can be configured to overcome standard forces acting on the guidewire (e.g., forces provided by blood flow rates of about 5 cm/sec to about 15 cm/sec). It will be appreciated that the bend 109 can overcome any force typically experienced by the insertion portion 104 within the patient's body.

The bend angle 112 can be defined as the angle between an axis 122 defined by the insertion portion 104 and a distal end 114 of the external portion 106 (e.g., the angle at which the external portion 106 is bent from a straight configuration). The bend angle 112 can be operable to overcome the force provided on the insertion portion 104 of the guidewire 100 during use. For example, as the insertion portion 104 is within the patient, the insertion portion 104 can be pulled further into the patient (e.g., within a blood vessel). The bend angle 112 can be operable to catch the skin of the patient or a dilator, catheter, or other instrument threaded on the guidewire 100, thereby preventing any further displacement of the insertion portion 104 into the patient. In this manner, the bend angle 112 can be operable to maintain the external portion 106 of the guidewire 100 outside of the patient without the need for a user to hold the external portion 106.

In some examples, the bend angle 112 can be about 45° or greater. It was surprisingly found that a bend angle 112 of at least 45° was operable to maintain the external portion 106 against the pulling force provided to the insertion portion 104 within the patient. In some examples, the bend angle 112 can be about 45° to about 180°. In some examples, the bend angle 112 can be at least 45°, at least 50°, at least 55°, at least 60°, at least 65°, at least 70°, at least 75°, at least 80°, at least 85°, at least 90°, at least 95°, at least 100°, at least 105°, at least 110°, at least 115°, at least 120°, at least 125°, at least 130°, at least 135°, at least 140°, at least 145°, at least 150°, at least 155°, at least 160°, at least 165°, at least 170°, at least 175°, at least 180°, or more. In some examples, the bend angle 112 can be about 45° to about 60°, about 60° to about 75°, about 75° to about 90°, about 90° to about 105°, about 105° to about 120°, about 120° to about 135°, about 135° to about 150°, about 150° to about 165°, about 165° to about 180°, or more. In some examples, the bend angle 112 can be about 80° to about 100°. In some examples, the bend angle 112 is about 90°. In some examples, the bend angle 112 can be about 180° to about 360° or more. For example, the bend angle 112 can be over 360° such that the external portion 106 coils on itself, as described further herein.

In some examples, the stopping mechanism 108 can include multiple bends. For example, as illustrated in FIG. 1D, the stopping mechanism 108 can include a second bend 111. In some examples, utilizing multiple bends can increase the safety of the guidewire 100. For example, if the bend 109 does not prevent retention of the guidewire 100, the second bend 111 can be operable to prevent retention of the guidewire 100. In this manner, the second bend 111 provides an additional level of guidewire retention prevention. It will be appreciated that any number of bends can be included on the guidewire 100.

In some examples, the bend 109 can be in any plane. For example, the bend 109 can extend in any direction (e.g., plane) from the insertion portion 104.

The bend radius 110 can be defined as the radius of the bend 109. The bend radius 110 can be operable to overcome the force provided on the insertion portion 104 of the guidewire 100. Further, the bend radius 110 can be operable to prevent kinking of the guidewire 100, such that the insertion portion 104 remains malleable. By maintaining the malleability of the insertion portion 104, the guidewire 100 can be navigated through the patient (e.g., through blood vessels) in substantially the same way as standard guidewires. Further, the bend radius 110 can allow the external portion 106 located after the bend 109 (e.g., from the end of the bend 109 to the distal end 114) to remain malleable. By maintaining portions or the external portion 106 as malleable, one or more instruments can be easily passed over the external portion 106 to the insertion portion 104.

In some examples, when the bend radius 110 is too small, the external portion 106 can become permanently kinked. When the external portion 106 becomes permanently kinked, the kink may prevent one or more instruments from passing over the external portion 106 to the insertion portion 104 (e.g., along the bend 109). Therefore, the bend radius 110 can be configured such that the external portion 106, and thereby the bend 109, remains semi-flexible such that one or more instruments can be passed over the bend 109. Further, when the bend radius 110 is too large, the bend 109 may not be able to maintain the external portion 106 outside of the patient in use (e.g., the guidewire 100 continues to displace into the body of the patient due to the force on the insertion portion 104 when the bend radius 110 is too large). For example, when the bend radius 110 is too large, the bend 109 may not be significantly rigid to hold the external portion 106 outside of the patient. Therefore, the bend radius 110 can be sized such that the bend 109 does not kink the catheter but also provides the necessary rigidity to overcome the force on the insertion portion 104.

In some examples, the bend radius 110 can be about 0.75 inches to about 2.50 inches. In some examples, the bend radius 110 can be greater than about 0.75 inches. In some examples, the bend radius 110 can be less than about 2.50 inches. In some examples, the bend radius 110 can be about 0.75 inches to about 1.00 inches, about 1.00 inches to about 1.25 inches, about 1.25 inches to about 1.50 inches, about 1.50 inches to about 1.75 inches, about 1.75 inches to about 2.00 inches, about 2.00 inches to about 2.25 inches, or about 2.25 inches to about 2.50 inches. In some examples, the bend radius 110 can be about 0.75 inches to about 1.25 inches. In some examples, the bend radius 110 can be about 1 inch.

In some examples, the bend 109 can be formed by wrapping the external portion 106 around a cylinder such that the bend 109 maintains the shape of the cylinder. For example, when the elongated wire 102 is a stainless steel core guidewire, the elongated wire 102 can be bent into shape thereby forming the bend 109. When the elongated wire 102 is a nitinol guidewire, the bend 109 can be formed by wrapping the external portion 106 around a cylinder having the intended bend radius and heating the external portion 106 to a sufficient temperature (e.g., 400° or higher) to form the bend 109. In other examples, guidewires with other materials can be used and other techniques can be used to form the bend 109.

In some examples, the external portion 106 can have a length 116 from the stopping mechanism 108 to the distal end 114. In some examples, the length 116 can be operable to allow a user to easily pass one or more instruments over the external portion 106 to the insertion portion 104. Further, the length 116 can be small enough such that the external portion 106 does not disrupt the maneuverability of the guidewire 100 (e.g., too long of a length can get in the way of the user). In some examples, the length 116 can be about 2 inches or more. In some examples, the length 116 can be about 2 inches to about 12 inches. In some examples, the length 116 can be any desired length.

FIG. 3 illustrates the guidewire 100 in another example. As illustrated in FIG. 3, the stopping mechanism 108 can include an expansion mechanism 208. The expansion mechanism 208 can be operable to be expanded such that the external portion 106 remains outside the patient. For example, the expansion mechanism 208 can be expanded and rest against the patient's skin to overcome the force provided to the insertion portion 104.

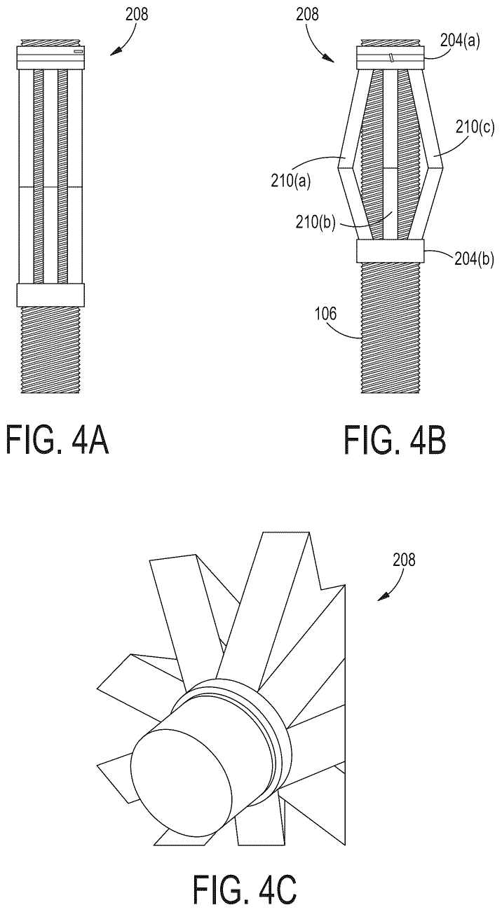

As illustrated in FIGS. 4A-4C, the expansion mechanism 208 can include a plurality of arms 210(a), 210(b), 210(c). The plurality of arms 210(a), 210(b), 210(c) can circumferentially surround a part of the external portion 106. The plurality of arms 210(a), 210(b), 210(c) can be attached to the external portion 106 using suitable coupling mechanisms 204(a), 204(b), such as clamps, snap-fit connectors, threaded connectors, magnetic connectors, friction fit connectors, spring-loaded mechanisms, interlocking tabs or hooks, latching mechanisms, bayonet connectors, rotary locking mechanisms, detent mechanisms, quick-release mechanisms, compression seals or gaskets, adhesive-based mechanisms, expandable collets or sleeves, push-button lock mechanisms, cam-locking mechanisms, and/or other types of coupling mechanisms. The coupling mechanisms 204(a), 204(b) can be operable to secure the plurality of arms 210(a), 210(b), 210(c) to the external portion 106.

The plurality of arms 210(a), 210(b), 210(c) can be operable to transition between a closed state, as illustrated in FIG. 4A, and an open state, as illustrated in FIG. 4C. In some examples, the plurality of arms 210(a), 210(b), 210(c), can begin in a semi-open state, as illustrated, for example, in FIG. 4B. Once the insertion portion 104 is within the body of the patient and a force is applied to the insertion portion 104, the plurality of arms 210(a), 210(b), 210(c) can be forced to expand in an accordion-like manner. In this manner, the plurality of arms 210(a), 210(b), 210(c) expand such that the plurality of arms 210(a), 210(b), 210(c) contact the skin of the patient and prevent the external portion 106 of the guidewire 100 from entering the patient.

The plurality of arms 210(a), 210(b), 210(c) can be operable to allow one or more instruments to be passed along the external portion 106 to the insertion portion 104. For example, a user can slide one or more instruments along the external portion 106 to the plurality of arms 210(a), 210(b), 210(c). The user can then provide a force to the one or more instruments (e.g., push the one or more instruments into the plurality of arms 210(a), 210(b), 210(c)), such that the plurality of arms 210(a), 210(b), 210(c) collapse to the closed state and the one or more instruments can be slid over the plurality of arms 210(a), 210(b), 210(c). Once the one or more instruments pass over the plurality of arms 210(a), 210(b), 210(c), the force is removed from the plurality of arms 210(a), 210(b), 210(c) and the plurality of arms 210(a), 210(b), 210(c) expand back to the semi-open state of FIG. 4B. Once the insertion portion 104 is displaced into the body by the force provided by the blood flow, the plurality of arms 210(a), 210(b), 210(c) contact the skin of the patient such that the plurality of arms 210(a), 210(b), 210(c) expand back to the open state of FIG. 4C. In this manner, the expansion mechanism 208 can be operable to allow one or more instruments to be passed along the external portion 106 to the insertion portion 104, while also preventing the external portion 106 from entering the body of the patient.

In other examples, the expansion mechanism 208 can include other types of expansion mechanisms operable to prevent guidewire retention. For example, the expansion mechanism 208 can include one or more of balloons, metallic arms, polymeric arms, spring-loaded prongs, collapsible meshes, expandable cages, inflatable cuffs, retractable hooks, magnetic clasps, compressible rings, flexible sleeves, interlocking plates, adhesive pads, sutures, clips, tension bands, elastic loops, ratcheting locks, helical coils, telescoping rods, interlocking jaws, or any combination thereof.

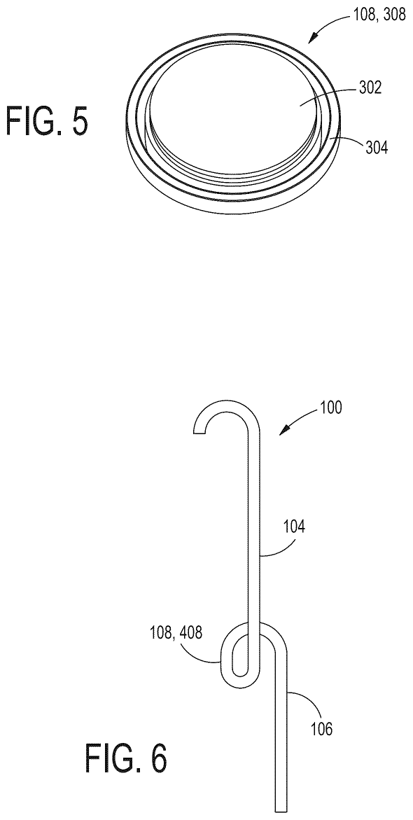

In some examples, as illustrated in FIG. 5, the stopping mechanism 108 can include a solid ring mechanism 308. The solid ring mechanism 308 can include a disc 302 and a ring surface 304. The solid ring mechanism 308 can be operable to receive the guidewire 100 through the disc 302. For example, the disc 302 can be operable to receive the guidewire 100 and subsequently secure the guidewire 100 within the disc 302. In some examples, the disc 302 can include a material operable to receive the guidewire 100 when a user pushes the guidewire 100 through the disc 302. In some examples, the material of the disc 302 can be elastic (e.g., an elastomer), such that the disc 302 is operable to open to receive the insertion portion 104 of the guidewire 100 and then close around the external portion 106 of the guidewire 100. In some examples, the material of the disc 302 can include an elastic foam and/or gel. A user can provide a sufficient force to the guidewire 100 to push the guidewire 100 through the disc 302. The force provided by the user can be a greater force than the force experienced by the insertion portion 104 within the user's body.

Once the disc 302 closes around the external portion 106 of the guidewire 100, the ring surface 304 can be operable to contact the patient's skin, thereby holding the external portion 106 outside of the patient. For example, the disc 302 can be operable to provide a sufficient friction force to the external portion 106 such that the force experienced by the insertion portion 104 is overcome by the friction force of the disc 302 grasping the external portion 106. In some examples, the disc 302 can have sufficient elasticity to allow the one or more instruments to be passed through the disc 302, and then the disc 302 regrasps the external portion 106 such that the external portion 106 is maintained outside the patient's body. For example, the one or more instruments can pass through the disc 302. In other examples, the one or more instruments can be passed along the external portion 106 to the insertion portion 104 prior to the solid ring mechanism 308 being located on the external portion 106.

In some examples, the stopping mechanism 108 can include a magnetic mechanism. For example, the stopping mechanism 108 can include a magnetic ring (e.g., similar to solid ring mechanism 308). The magnetic ring can be operable to exert a magnetic force on the external portion 106 of the elongated wire 102 such that the external portion 106 is held outside of the patient's body. For example, the external portion 106 can include a magnetic element that attracts to the magnetic ring such that the magnetic element of the external portion 106 is held outside of the patient's body.

In some aspects, the stopping mechanism 108 can include an adhesive mechanism. For example, the stopping mechanism 108 can include be an adhesive material that sticks to the external portion 106 of the elongated wire 102. The adhesive mechanism can have a diameter sized to prevent retention of the guidewire 100. For example, the adhesive mechanism can be operable to catch on the patient's skin or a dilator, catheter, or other instrument guided by the guidewire 100, such that the external portion 106 of the guidewire 100 is prevented from slipping into the patient.

In some examples, as illustrated in FIG. 6, the stopping mechanism 108 can include a coil 408 (e.g., coiled section) in the external portion 106 of the elongated wire 102. In some examples, the coil 408 can be operable to rest on the patient's skin while the insertion portion 104 is within the patient's body. For example, the coil 408 can be operable to maintain the external portion 106 outside of the patient's body. In some examples, the coil 408 can have a diameter greater than the blood vessel of the patient, such that the coil maintains the external portion 106 outside of the patient's body. In some examples, the coil 408 can allow for maneuverability of one or more instruments around the coil 408 (e.g., providing a sufficient force can flatten the coil 408 such that one or more instruments can be moved through the coil 408). The coil 408 can bias such that when a significant force (e.g., greater than the force exerted on the insertion portion 104 by the patient's body) is removed the coil 408 springs back to its shape.

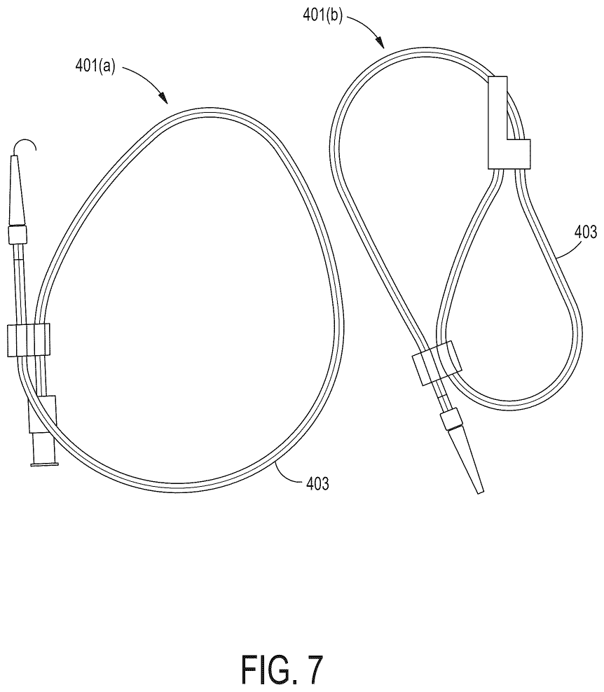

Further provided herein is a kit for storing the guidewire 100. FIG. 7 illustrates a standard guidewire kit 401(a) and a kit 401(b) modified to store the guidewire 100 described herein. The standard guidewire kit 401(a) includes a tube 403 that has a substantially circular configuration. The kit 401(b) can be operable to store the guidewire 100 with the bend 109 described herein. For example, the kit 401(b) can include a tube 403 that is bent in substantially the same shape as the bend 109. By storing the guidewire 100 with the bend 109 in substantially the same shape as the bend 109, the bend 109 can maintain its shape for a prolonged period of time (e.g., days, months, or years). In this manner, the bend 109 can already be in place when a user is ready to use the guidewire 100.

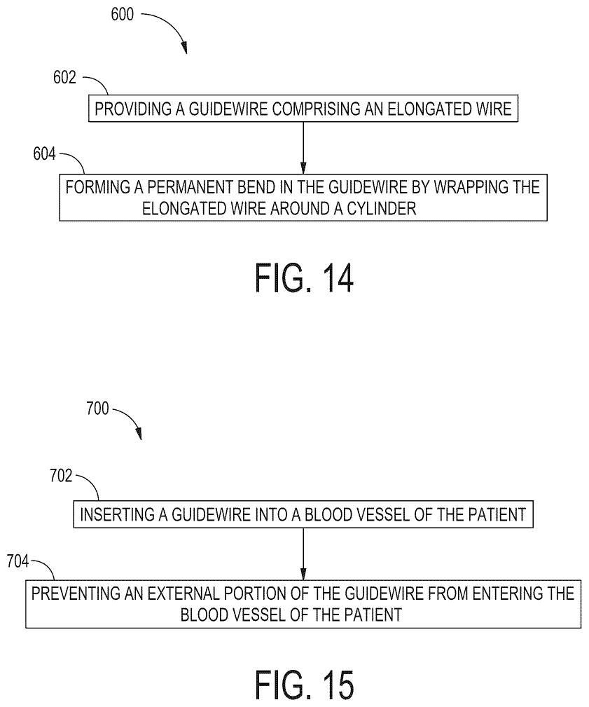

Further provided herein is a method for manufacturing the guidewire described herein. FIG. 14 illustrates a method 600 for manufacturing the guidewire with the bend described herein.

At block 602, the method 600 can begin by providing a guidewire comprising an elongated wire. For example, the elongated wire can have an external portion and an insertion portion, as described herein.

At block 604, the method 600 can include forming a bend in the guidewire by wrapping the elongated wire around a cylinder. In some examples, the external portion of the elongated wire can be wrapped around the cylinder (e.g., the bend is located along the external portion). In some examples, the bend can have a bend angle of about 45 degrees or greater and a bend radius of between about 0.75 inches and 2.50 inches. In some examples, the bend can prevent the external portion from entering a blood vessel of the patient. For example, as described herein, the bend can be operable to contact the skin of the patient and overcome a force provided to the insertion portion (e.g., a force provided from the blood flow of the patient when the insertion portion is within a blood vessel).

In some examples, the bend radius can be about 0.75 inches to about 2.50 inches. In some examples, the bend radius can be greater than about 0.75 inches. In some examples, the bend radius can be less than about 2.50 inches. In some examples, the bend radius 110 can be about 0.75 inches to about 1.00 inches, about 1.00 inches to about 1.25 inches, about 1.25 inches to about 1.50 inches, about 1.50 inches to about 1.75 inches, about 1.75 inches to about 2.00 inches, about 2.00 inches to about 2.25 inches, or about 2.25 inches to about 2.50 inches. In some examples, the bend radius can be about 0.75 inches to about 1.25 inches. In some examples, the bend radius can be about 1 inch.

In some examples, the bend angle can be about 45° or greater. In some examples, the bend angle can be about 45° to about 180°. In some examples, the bend angle can be at least 45°, at least 50°, at least 55°, at least 60°, at least 65°, at least 70°, at least 75°, at least 80°, at least 85°, at least 90°, at least 95°, at least 100°, at least 105°, at least 110°, at least 115°, at least 120°, at least 125°, at least 130°, at least 135°, at least 140°, at least 145°, at least 150°, at least 155°, at least 160°, at least 165°, at least 170°, at least 175°, at least 180°, or more. In some examples, the bend angle can be about 45° to about 60°, about 60° to about 75°, about 75° to about 90°, about 90° to about 105°, about 105° to about 120°, about 120° to about 135°, about 135° to about 150°, about 150° to about 165°, about 165° to about 180°, or more. In some examples, the bend angle can be about 80° to about 100°. In some examples, the bend angle is about 90°.

In some examples, the cylinder can have a formation bend radius. In some examples, the formation bend radius can be less than the bend radius of the bend. In some examples, wrapping the elongated wire around a cylinder having a formation bend radius less than the bend radius of the desired bend allows the elongated wire to experience a spring back (e.g., bounce back) while still forming the desired bend. In some examples, the formation bend radius can be about 0.1 inches to about 0.5 inches less than the desired bend radius of the bend.

In some examples, when the elongated wire is a stainless steel core wire, wrapping the elongated wire around the cylinder is sufficient to form the bend (e.g., the elongated wire substantially maintains the shape of the bend when removed from the cylinder). In other examples, the elongated wire can be a nitinol wire. The nitinol wire may require heat treatment for forming the bend. For example, the nitinol wire can be wrapped around the cylinder and then heated to a shape setting temperature. In some examples, the shape setting temperature can be about 400° Celsius or greater. In some examples, once the nitinol wire is wrapped around the cylinder at the desired bend angle and bend radius, the nitinol wire and cylinder can be heated in a box furnace or via electrical resistance techniques to the shape setting temperature for a period of time, thereby forming the bend in the nitinol wire. In some examples, the period of time can be about 1 minute to about 10 minutes. Once the bend is formed, the nitinol wire can be removed from the heat and can be ready for use in a patient. In some examples, the elongated wire can include other materials, and the bend can be formed using other techniques.

Further provided herein is a method for using the guidewire described herein. FIG. 15 illustrates a method 700 for using the guidewire described herein. At block 702, the method 700 can include inserting a guidewire into a blood vessel of the patient. Inserting the guidewire into the blood vessel can include first inserting a hollow needle into the blood vessel. An insertion portion of the guidewire can then be threaded through the hollow needle, into the blood vessel, and to a target site within the blood vessel.

At block 704, the method 700 can include preventing an external portion of the guidewire from entering the blood vessel of the patient. For example, any of the stopping mechanisms described herein can be used to prevent the external portion of the guidewire from entering the blood vessel. The stopping mechanism can include the bend described herein, the expansion mechanism described herein, the solid ring mechanism described herein, or the coil described herein. The stopping mechanism can be operable to overcome a force provided to the insertion portion of the guidewire. For example, the blood flow of the patient can provide a force to the insertion portion, thereby pulling the insertion portion further into the blood vessel. The stopping mechanism can be operable to overcome the force provided by the blood flow, thereby maintaining the external portion of the guidewire outside of the patient. In this manner, guidewire retention and embolization are prevented.

In some examples, the method 700 can further include removing the hollow needle from the target site. For example, the hollow needle can be removed from the target site by pulling the hollow needle along the insertion portion of the guidewire, through, or over, the stopping mechanism, and off the external portion of the guidewire.

In some examples, the method 700 can further include providing one or more instruments to the target site within the blood vessel. For example, the one or more instruments can include catheters, dilators, needles, and other medical devices. The one or more instruments can be provided to the target site by translating the one or more instruments along the external portion, through, or over, the stopping mechanism, and to the insertion portion of the guidewire.

In some examples, the method 700 can further include performing one or more medical operations with the one or more instruments. Any medical operation typically performed by instruments located within a patient using a guidewire can be performed with the method 700.

In some examples, the method 700 can further include removing the guidewire. In some examples, removing the guidewire can include grasping the external portion of the guidewire and pulling the guidewire out of the blood vessel.

### EXAMPLES

### Example 1

The guidewire 100 described herein was tested under conditions simulating the venous blood flow of a patient. Techniques for insertion of a guidewire into a blood vessel of a patient assume the risk of the guidewire being drawn into the vein and causing complications (e.g., perforating the vascular wall, arrhythmias, infection, blood clots, perforation, lung and heart damage). This concern results in the precautionary practice of never letting go of the guidewire when it is used within a patient.

The guidewire 100 described herein seeks to alleviate this concern mitigating the risk of the guidewire 100 being drawn into a vein. This can be accomplished by altering the shape of the guidewire to complicate drawing the guidewire into the vein. This study aims to determine the limits of the bend angle and the bend radius required to prevent the guidewire 100 from being drawn into the patient, while also maintaining the ability to be inserted into the vein and the structural integrity of the guidewire 100.

The objectives of this study were (1) determine the maximum radius of the geometry of the bend in the guidewire to arrest movement of the guidewire into the vein, (2) determine the minimum radius of the geometry of the bend in the guidewire without compromising the structural integrity of the guidewire, and (3) determine the minimum degrees of curvature required arrest movement of the guidewire into the vein.

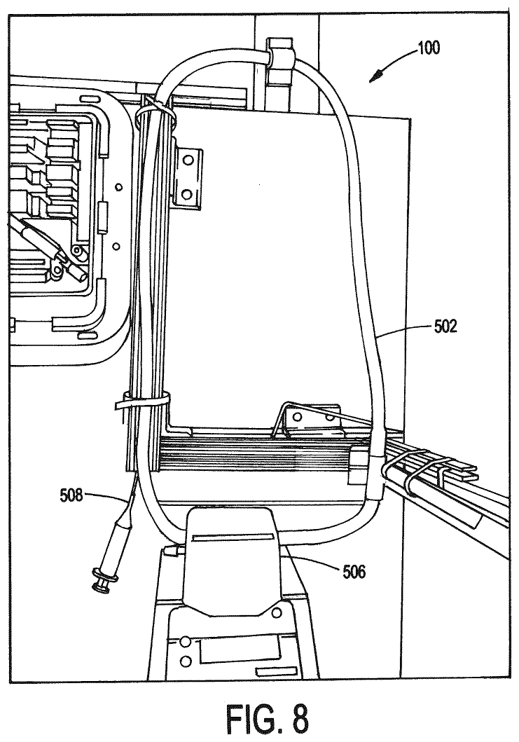

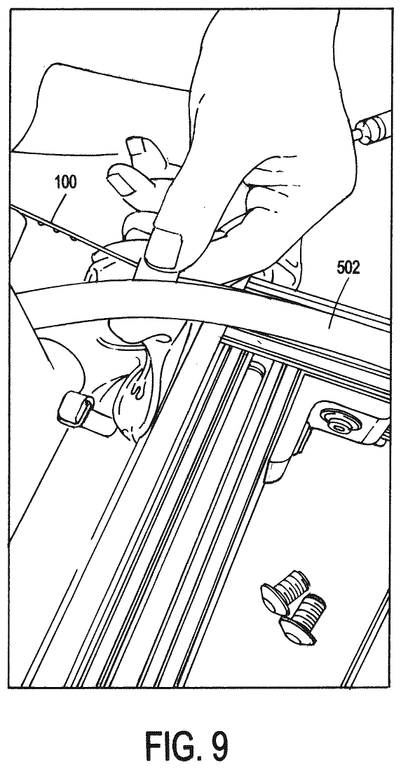

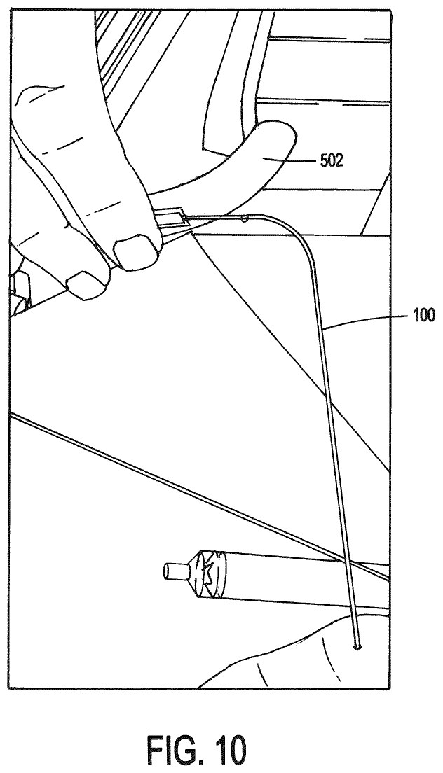

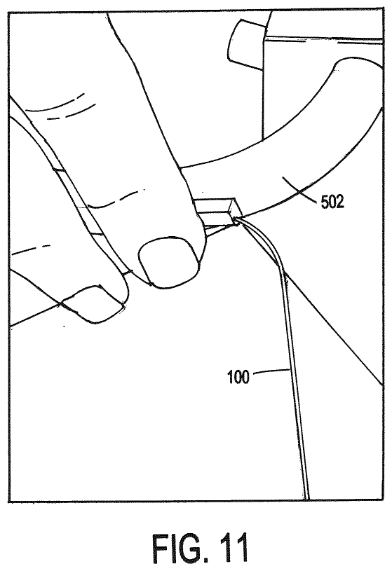

FIGS. 8-11 illustrate the test apparatus 500. The test apparatus includes a tube 502 in fluid communication with a pump 506. The pump 506 is operable to pump liquid through the tube 502 such that the flow rate within the tube 502 substantially simulates blood flow in a patient. The test apparatus further includes a needle 508 operable to receive the guidewire 100, thereby placing the guidewire 100 within the flow of liquid in the tube 502. The parameters of the liquid for the tests are shown in Table 1.

TABLE 1
Liquid Parameters

| Condition | Value | |
|---|---|---|
| Pressure | 8 mmHg or 696 Pascals | |
| Flow Rate | 45 | cm/sec |
| Viscosity | 0.4 | Pa-S |

The guidewire 100 was tested at 100% of the values of Table 1. The guidewire 100 was also tested at 175% of the pressure and flow rate of Table 1 to ensure conservative results.

To establish the maximum function bend radius, a battery of tests was performed on guidewires with increasing large bend radii. As illustrated in Table 2, a baseline bend angle of 90° was used for bend radius portion of the test. As illustrated in Table 3, a baseline bend radius of 1 inch was used for the bend angle portion of the test. These values were chosen for ease of use and verification for the intended user and because the values performed well in all preliminary and protype tests. The dataset of Table 2 is incomplete. However, extrapolating the data set provides the conclusion that tests 5-9 would be satisfactory and have an acceptable shape. Similarly, the bend radii greater than or equal to test 11 would be unsatisfactory.

TABLE 2
Test Results - Bend Radius

| | Bend | Degrees | Successful | |
|---|---|---|---|---|
| Test | Radius | of Bend | Stop | Structural Integrity |
| 1 | 0.25″ | 90°+ | Yes | Kinked beyond repair, |
| | | | | difficult to insert |
| | | | | into tube |
| 2 | 0.50″ | 90°+ | Yes | Kinked beyond repair, |
| | | | | inserts with little |
| | | | | difficulty |
| 3 | 0.75″ | 90°+ | Yes | Article remains |
| | | | | malleable, inserts |
| | | | | easily into the tube |
| 4 | 1.00″ | 90°+ | Yes | Article remains |
| | | | | malleable, inserts |
| | | | | easily into the tube |
| 5 | 1.25″ | 90°+ | | |
| 6 | 1.5″ | 90°+ | | |
| 7 | 1.75″ | 90°+ | | |
| 8 | 2.00″ | 90°+ | | |
| 9 | 2.25″ | 90°+ | | |
| 10 | 2.50″ | 90°+ | Yes | Article remains |
| | | | | malleable, inserts |
| | | | | easily into the tube |
| 11 | 2.75″ | 90°+ | No | Article remains |
| | | | | malleable, inserts |
| | | | | easily into the tube |
| 12 | 3.00″ | 90°+ | | |

TABLE 3
Test Results - Bend Angle

| Test | Bend Radius | Degrees of Bend | Successful Stop |
|---|---|---|---|
| 13 | 1.00″ | 10°+ | No |
| 14 | 1.00″ | 25°+ | No |
| 15 | 1.00″ | 45°+ | Yes |
| 16 | 1.00″ | 75°+ | Yes |
| 17 | 1.00″ | 85°+ | Yes |

The test apparatus 500 was set up to both realistically mimic the conditions and environment experienced by the guidewire and to go above and beyond those values to create a factor of safety threshold at 175% of the worst-case scenario for these values. The results of these tests were that a bend angle of at least 45° and a bend radius of between 0.75 inches and 2.50 inches are required for a guidewire that reliably arrests motion due to suction in a vein and maintains structural integrity. A 90°+ bend angle with a radius of 0.75 inches to 1.00 inches is a good candidate for development due to the guidewire's strong stopping ability and ease of verification for the end user.

The guidewire 100 was manufactured using the methods described herein. For example, the tests were conducted using a stainless-steel core guidewire. The guidewire 100 was wrapped tightly around a cylinder of lesser radius than the intended shape to account for the spring back. The method can be reliably repeated to manufacture consistent guidewire geometries.

Some of the guidewires did not have a stainless-steel core. Rather, these guidewires have a nitinol core and could not be shaped through the normal bending process. Nitinol is shaped with heat. Nitinol guidewires can be heated and formed into the desired guidewire geometry. For example, the guidewire can be wrapped around the intended diameter and heated in a box furnace or via electrical resistance techniques to its shape setting temperature. The shape setting temperature can be 400° C. or greater for about 1 minute to about 10 minutes. The guidewire will then hold this shape when removed from heat.

### Example 2

The guidewire 100 was pull tested to failure to determine if the modification to the wire had detrimental impacts to the tensile strength of the guidewire 100. The weld at the unmodified stock J-hook broke at 4.56 lbs static load before the bend 109 broke.

### Example 3

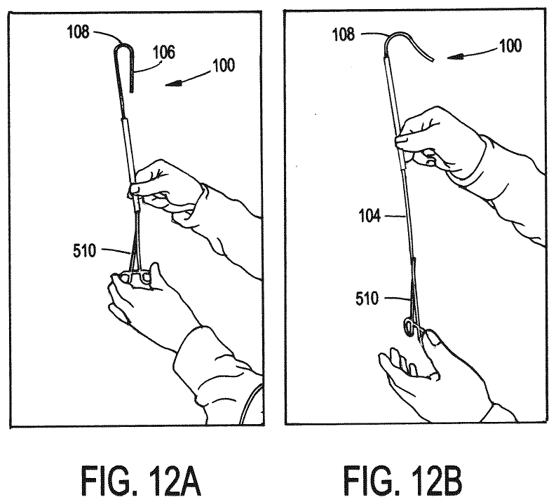

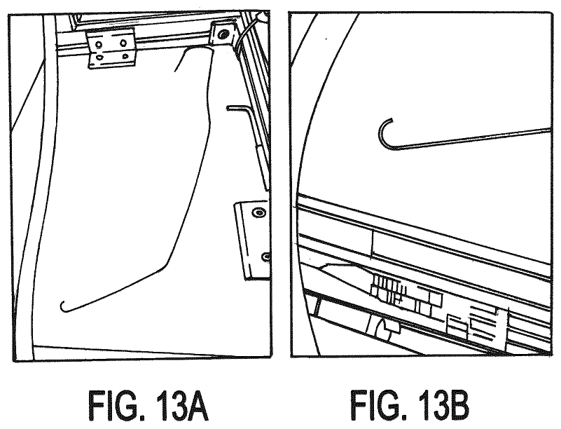

The guidewire 100 was tested using a gravity test, as illustrated in FIGS. 12A-12B. The guidewire was inserted into a tube with the stopping mechanism 108 above the tube, as illustrated, for example, in FIG. 12A. The insertion portion 104 of the guidewire was attached to a 35 gram load 510. The load was then released. The external portion 106 of the guidewire 100 remained above the tube due to the stopping mechanism 108, as illustrated in FIG. 12B.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A guidewire for preventing guidewire retention comprising:
an elongated wire comprising an insertion portion and an external portion; and
a stopping mechanism located along the external portion, the stopping mechanism configured to prevent the external portion from entering a blood vessel of a patient.

2. The guidewire of claim 1, wherein the stopping mechanism comprises a bend.

3. The guidewire of claim 2, wherein the bend has a bend angle between the insertion portion and the external portion.

4. The guidewire of claim 3, wherein the bend angle is at least 45 degrees.

5. The guidewire of claim 3, wherein the bend angle is at least 90 degrees.

6. The guidewire of claim 3, wherein the bend has a bend radius of greater than about 0.75 inches.

7. The guidewire of claim 6, wherein the bend radius is less than about 2.50 inches.

8. The guidewire of claim 6, wherein the bend radius is about 1 inch.

9. The guidewire of claim 2, wherein the bend is heat treated.

10. The guidewire of claim 1, wherein the stopping mechanism comprises a coiled section of the elongated wire.

11. The guidewire of claim 1, wherein the stopping mechanism comprises an expansion mechanism, an adhesive mechanism, and/or a magnetic mechanism attached to the elongated wire.

12. The guidewire of claim 1, wherein the stopping mechanism comprises a solid ring mechanism operable to receive the elongated wire and secure to a location on the external portion.

13. The guidewire of claim 1, wherein the elongated wire comprises stainless steel.

14. The guidewire of claim 1, wherein the elongated wire comprises nitinol.

15. A method of manufacturing a guidewire, the method comprising:
providing the guidewire comprising an elongated wire; and
forming a bend in the guidewire by wrapping the elongated wire around a cylinder, wherein the bend has a bend angle of 45 degrees or greater and a bend radius of between about 0.75 inches and 2.50 inches,
wherein the elongated wire comprises an insertion portion and an external portion,
wherein bend is located along the external portion, and
wherein the bend prevents the external portion from entering a blood vessel of a patient.

16. The method of claim 15, wherein the bend radius is about 1 inch.

17. The method of claim 15, wherein the bend angle is 90 degrees or greater.

18. The method of claim 15, wherein forming the bend further comprises heating the bend.

19. The method of claim 15, wherein the elongated wire is wrapped around the cylinder at a formation bend radius.

20. The method of claim 19, wherein the formation bend radius is less than the bend radius, thereby allowing the elongated wire to bounce back to the bend radius.
